(19) United States
(12) Patent Application Publication
(10) Pub. No.: US 2002/0049431 A1
(43) Pub. Date: Apr. 25, 2002

(54) Method of correcting corneal refraction not aligned with the pupil center

(76) Inventors: Michael J. Smith, Orlando, FL (US); Lance R. Marrou, Orlando, FL (US)

Correspondence Address: MANELLI DENISON & SELTER PLLC, 7th Floor, 2000 M Street, N.W., Washington, DC, 20036-3307, US

(21) Appl. No.: 09/982,960

(22) Filed: Oct. 22, 2001

Related U.S. Application Data
(60) Provisional application No. 60/241,752, filed on Oct. 20, 2000.

(51) Int. Cl. A61B 18/18

(52) U.S. Cl. 606/5

(57) ABSTRACT

A method is provided of removing less corneal tissue during refractive surgery, when a visual axis or optical axis of an eye is not aligned with a pupil center. The method determines an initial keratometry surface around the visual axis. A visual system error is determined and the visual system error is applied to the initial keratometry surface to determine a target keratometry surface. The initial and target keratometry surfaces are offset by an amount substantially equal to an offset of the visual axis from the pupil center. The target keratometry surface is subtracted from the initial keratometry surface to establish values along a chosen corneal treatment zone diameter. All the values along the chosen corneal treatment zone diameter and within it are set to be positive, thereby defining a resulting ablation profile. The resulting ablation profile is used as a guide for corneal tissue to remove, with greater positive values defining deeper ablation depths.

This application is based on and claims priority from U.S. Provisional Application No. 60/241,752 filed on Oct. 20, 2000, the entirety of which is expressly incorporated herein by reference.

### FIELD OF THE INVENTION

The invention relates to corneal refractive corrections and, more particularly, to a method of adjusting the amount of tissue removed from a cornea during refractive surgery, when the visual axis is not coincident with the pupil center. This also applies when the optical axis (of the visual system) is not aligned with the visual axis (the axis of the line of sight).

### BACKGROUND OF THE INVENTION

For corneal refractive surgery, an important feature is the patient's pupil size. The human pupil is the aperture of the eye. Tissue is removed during refractive surgery in order to resculpt the cornea and correct for errors in vision caused by a non-optimized corneal curvature. The cornea is corrected to the outer diameter of a dilated pupil in order to provide improved correction. If the dilated scotopic pupil is not used as the outside diameter for correction (optical zone), then night vision problems, glare, or visual halos may occur. For patients with larger pupil sizes, a larger amount of corneal tissue is removed to prevent glare or halos that may be caused by partially seeing through uncorrected locations of the cornea. The photopic pupil size might also be considered, but it will be less than the scotopic pupil so if the surgery accommodates the scotopic pupil then it will also accommodate the photopic pupil.

The human eye does not require corrections that exceed the size of the scotopic pupil and therefore any cornea correction beyond this is generally unneeded. However, some tissue may be removed outside the optical zone in order to provide a smooth transition between treated and untreated cornea. Since corneal refractive correction involves removal of tissue, any increase in the area of correction will normally be associated with more tissue removed. In general, the less tissue removed, the better it is for the patient and the shorter the treatment time. Since there is a clinically accepted maximum safe depth of corneal tissue removal, limiting the depth is a major consideration when planning a refractive surgery.

In the human optical system, the center of the pupil and the location through which the person looks (referred to as the visual axis) are not always coincident. For some patients, the difference between these two points can be as much as 600 microns. Current systems that track the pupil to center the treatment can suffer the problem of misalignment with the patient's visual axis. To correct this problem, the entire ablation profile (tissue to be removed from the cornea to reshape it to the ideal) is shifted from the pupil center by an apriori known difference between the visual axis and pupil center, and enlarged to encompass the entire pupil (i.e., optical zone). This simple, yet intuitive solution introduces a problem. If the surgeon is still to correct the entire pupil area, the size of the ablation (and the corresponding depth) will have to be increased by the amount of the offset. For example, FIG. 5A shows a conventional ablation area when the pupil center is offset from the visual axis. This leads to corneal tissue that is removed which is not over the pupil and increased tissue removal depth for the larger treatment area as shown in FIG. 5B. The computation of the ablation may also account for the optical axis, but the shift of the ablation is still relative to the pupil center, because the ablation should always cover the entire pupil.

FIG. 6 shows a portion of an eye exhibiting an exemplary offset between a visual axis, optical axis, and a pupil center of an eye.

### SUMMARY OF THE INVENTION

An object of the invention is to decrease the total amount of tissue removed during a corneal refractive surgery. In accordance with the principles of the present invention, this objective is achieved by providing a method of identifying corneal tissue to be ablated during refractive surgery, when a visual axis or optical axis of an eye is not aligned with a pupil center. The method determines an initial keratometry surface around the visual axis. A visual refractive error around the visual or optical axis is determined and the visual refractive error is applied to the initial keratometry surface to determine a target keratometry surface. The initial and target keratometry surfaces are offset by an amount equal to offset of the visual or optical axis from the pupil center. The target keratometry surface is subtracted from the initial keratometry surface to establish values along a chosen corneal treatment zone diameter. All the values along the chosen corneal treatment zone diameter and within it are set to be positive, thereby defining a resulting corneal tissue removal profile (referred to as an ablation profile). The resulting ablation profile is used as a guide for corneal tissue to remove, with greater positive values defining deeper ablation depths.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, in which:

FIG. 1 is a view of a portion of an eye showing an exemplary offset between a visual axis and a pupil center of an eye, and corneal tissue to be removed in accordance with the invention. The optical axis is aligned with the visual axis.

FIG. 2A is a plan view showing an ablation area centered about the pupil center and a refractive correction area centered about the visual axis, in accordance with the invention.

FIG. 2B is a view showing depth of tissue ablation of the ablation area of FIG. 2A.

FIG. 3 is a view of an ablation profile in accordance with the invention, wherein the center of the ablation correction is offset from the pupil center.

FIG. 4 is a flow chart of a method of the present invention of reducing tissue removed during a corneal refractive surgery when the visual axis is offset from the pupil center.

FIG. 5A is a schematic plan view of a conventional corneal tissue ablation area when the pupil center is offset from the visual axis.

FIG. 5B is a view showing depth of tissue ablation of the ablation area of FIG. 5A.

FIG. 6 is a view of a portion of an eye showing an exemplary offset between a visual axis, optical axis, and a pupil center of an eye.

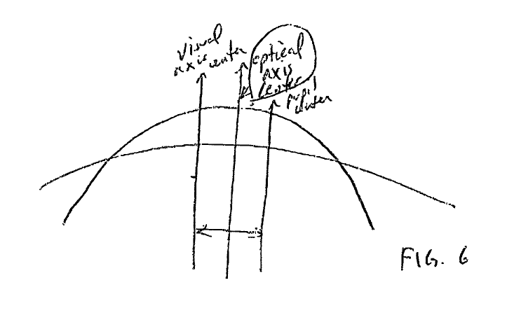

### DETAILED DESCRIPTION OF THE PREFERRED EMBODIMENT

Each human cornea has a given shape that defines where the eye system focuses. There are devices (such as phoropters) that optometrists use for determining proper glasses that determine the error in the eye's focal distance. This invention relates to refractive surgery which works by removing material from the cornea to shape it into a surface that corrects for this refractive focal distance error.

An important consideration for this surgery is how big the corrected area of the cornea is. Just like having glasses with very small lenses, having a small treatment zone (the area of affected corneal tissue) can create glare, halos, or fuzziness at the edge of images if the treated zone is less than the viewing pupil diameter. For this reason, surgical refractive devices allow the surgeon to pick a treatment zone size that is big enough to cover the entire visual area (normally this means making the treatment zone the size of the largest pupil size during viewing).

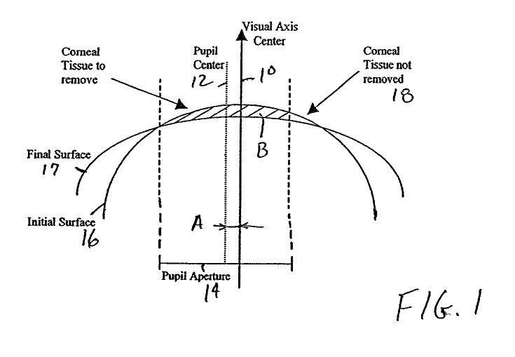

In refractive surgery, optimally, a treatment centered on the pupil would be performed and measured error would be corrected. However, the accuracy of this treatment is unlikely, since, in normal human eyes, the human visual system does not look through the center of the pupil. In fact, with reference to FIG. 1, there is normally a difference (A) between where the visual system looks out (visual axis 10) and the pupil center 12. Some people having severe problems wherein their visual axis 10 is greatly different from their pupil center 12 suffer the anomaly of being able to see people when they appear to not be looking at them. This is an extreme example of the potential affects of a significant difference between centers of the pupil and visual axes.

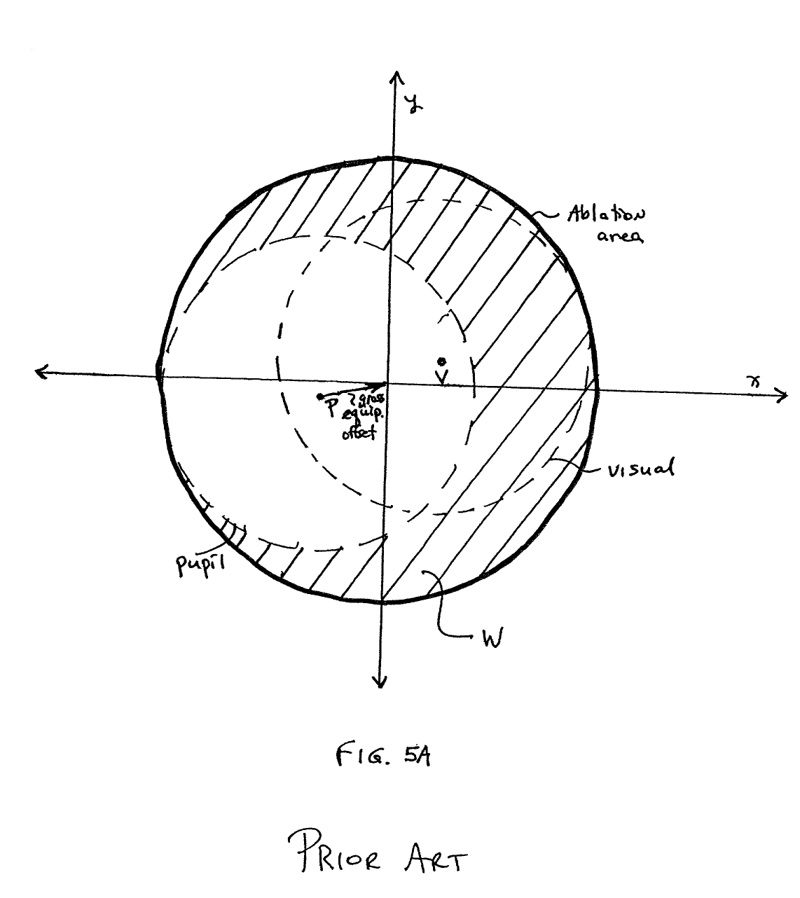

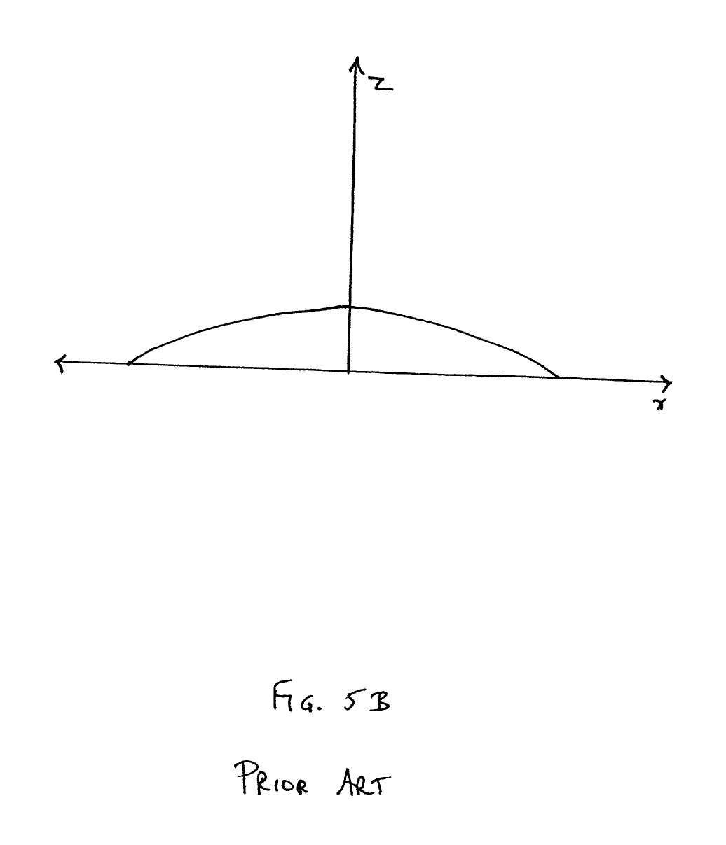

To properly treat the refractive error for any patient in accordance with the preset invention, the change or sculpting is centered on the visual axis 10. Since ideally the entire pupil 14 is to be treated, this creates a separate constraint. The conventional solution is to center the ablation on the visual center with a treatment area that is large enough to encompass the entire pupil aperture 14, as shown by the ablation area of FIG. 5A. This method works, but results in removal of excess tissue that does not improve the patient's vision.

The present invention provides an ablation which is centered on the visual axis, but which also minimizes the amount of corneal tissue removed.

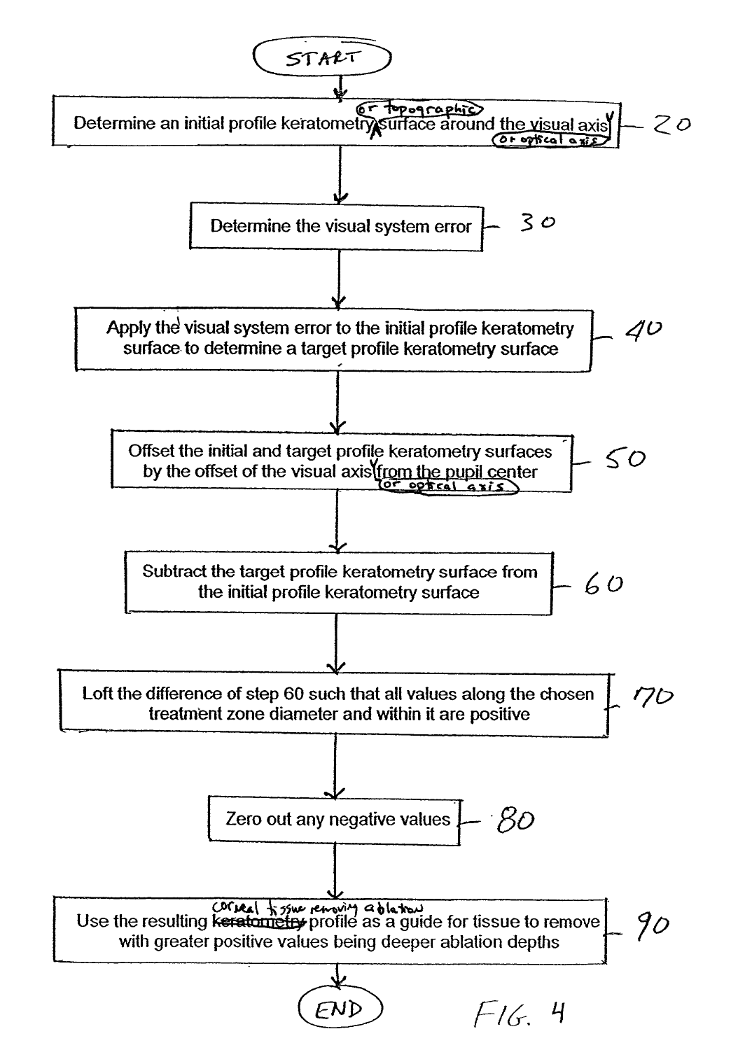

FIG. 4 is a flow chart of a method of the present invention of reducing tissue removed during a corneal refractive surgery when the visual axis 10 is offset from the pupil center 12. In particular, with reference to step 20 FIG. 4, an initial profile keratometry surface around the visual axis 10 is determined. Typically, this is a value determined from a keratometer or topographic keratometer and is shown as the initial keratometry surface 16 in FIG. 1. This may also be the initial topography of the eye.

In step 30, a visual system error is determined. This can be done by using a phoropter or auto-refractor device to determine the refractive error and is a difference between the initial keratometry surface 16 and a target or final keratometry surface 17 (FIG. 1).

In step 40, the visual system error is applied to the initial keratometry surface 16 to determine the target keratometry surface or final keratometry surface 17. This step involves adding the visual system error to the initial keratometry surface 16. The method of adding the visual system error to the initial keratometry surface can be as simple as arithmetic addition (e.g. 44D initial keratometry plus a −5D refractive error equals a 39D final keratometry surface), or as complex as required for the given keratometry source and desired change in refraction, including asphericity, regular and irregular astigmatism, or other visual system errors attributed from wavefront or other diagnostic devices.

In step 50, the initial keratometry surface 16 and the target or final keratometry surface 17 are offset by an amount substantially equal to an offset of the visual axis 12 from the pupil center 12 (e.g., the distance A in FIG. 1).

In step 60, the target or final keratometry surface 17 is subtracted from the initial keratometry surface 16 to define an area between the initial and final keratometry surfaces. This area is the entire area between the final keratometry surface 17 and the initial keratometry surface 16, including corneal tissue not removed 18.

In step 70, the difference determined in step 60 is lofted such that all values along a chosen treatment zone diameter and within it are positive values.

In step 80, any negative values are zeroed-out including values outside the pupil aperture 14. Thus, values in the area defined by corneal tissue 18, not removed, are zeroed-out.

In step 90, the resulting ablation profile is used as a guide for tissue to remove, with greater positive values being deeper ablation depths. Thus, with reference to FIG. 1, the cross-hatched area B is removed to obtain the final surface 17, with tissue 18 remaining.

The method steps of FIG. 4 are preferably implemented in appropriate software, e.g., in a controller of a corneal laser ablation system.

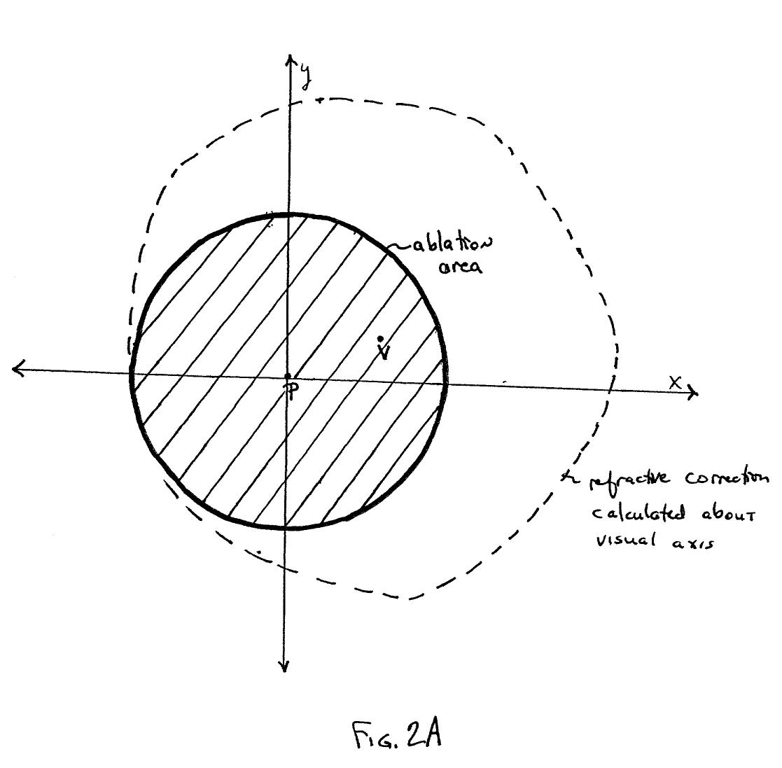

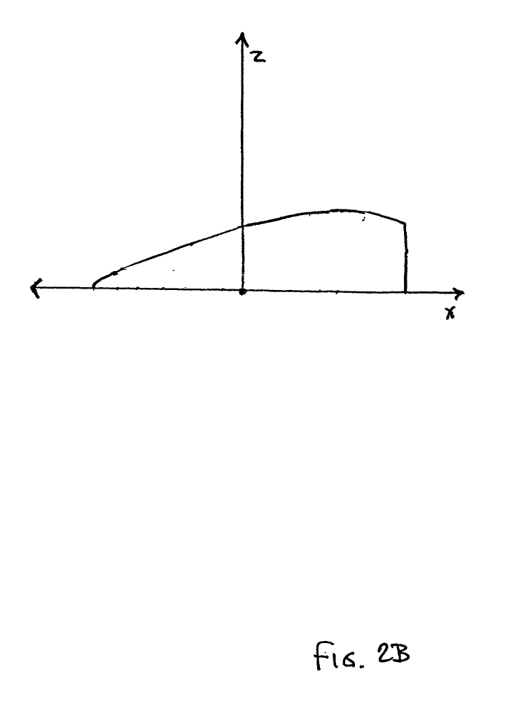

Thus, in comparing the depth of ablation that results with the method of the invention as shown in FIG. 2 with the depth of ablation of the conventional ablation method as shown in FIG. 5B, it can be seen that less tissue is removed by the method of the invention.

In addition, with the method of the invention as illustrated in FIG. 2A, the ablation area is always centered on the pupil, but the refractive correction is centered, or calculated, about the visual axis V which is offset from the pupil center P.

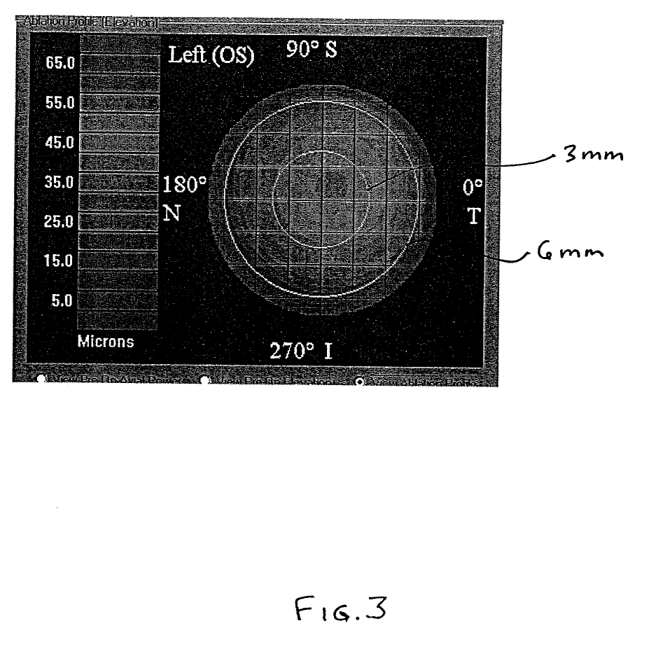

FIG. 3 is a view of an ablation profile in accordance with the invention, wherein the center of the ablation correction is offset from the pupil center.

In particular, as seen in FIG. 3, the deepest portion of the ablation correction is shown above and to the right of the center of the pupil (on which the correction is centered). However, importantly, the ablation does not exceed significantly the boundary defined by the 6 mm diameter circuit corresponding to the pupil. Thus, an asymmetrical ablation results about the center of the ablation pattern.

The above-defined technique works similarly for topography data that is visually axis aligned. For instance, the topography data may be offset so that the pupil becomes the center of the data map. When this technique is applied, the desired correction similar to that resulting from conventional axis alignment techniques but with the removal of significantly less corneal tissue.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A method of identifying corneal tissue to be ablated, said method comprising:
determining an initial surface around a visual axis of an eye;
determining a visual system error of said eye;
applying said visual system error to said initial surface to determine a target surface;
offsetting said initial surface and said target surface each by an amount substantially equal to an offset of said visual axis from said pupil center; and
subtracting said target surface from said initial surface to establish ablation values.

2. The method of identifying corneal tissue to be ablated according to claim 1, further comprising:
locating said visual axis of said eye.

3. The method of identifying corneal tissue to be ablated according to claim 2, further comprising:
locating said pupil center of said eye.

4. The method of identifying corneal tissue to be ablated according to claim 1, wherein said initial surface and said target surface each comprise:
a keratometric surface.

5. The method of identifying corneal tissue to be ablated according to claim 1, wherein said initial surface comprises:
a wavefront surface.

6. The method of identifying corneal tissue to be ablated according to claim 1, wherein:
said wavefront surface is a piecewise refractive surface.

7. The method of identifying corneal tissue to be ablated according to claim 1, wherein:
said established ablation values are determined along a diameter of a chosen corneal treatment zone.

8. The method of identifying corneal tissue to be ablated according to claim 7, further comprising:
ensuring that said established ablation values along said diameter of said chosen corneal treatment zone and within said diameter are positive thereby defining a resulting ablation profile.

9. The method of identifying corneal tissue to be ablated according to claim 8, further comprising:
using said resulting ablation profile as a guide for corneal tissue to be ablated, with greater positive values defining deeper ablation depths.

10. The method of identifying corneal tissue to be ablated according to claim 8, wherein said step of ensuring comprises:
setting to zero any negative ones of said established ablation values.

11. The method of identifying corneal tissue to be ablated according to claim 1, wherein:
said step of determining said initial surface utilizes a topographic keratometer.

12. The method of identifying corneal tissue to be ablated according to claim 1, wherein:
said step of determining said visual system error utilizes a phoropter.

13. The method of identifying corneal tissue to be ablated according to claim 1, wherein:
said step of determining said visual system error utilizes an auto-refractor device.

14. The method of identifying corneal tissue to be ablated according to claim 1, wherein said step of applying said visual system error to said initial surface comprises:
adding said visual system error to said initial surface.

15. The method of identifying corneal tissue to be ablated according to claim 7, wherein:
said diameter of said chosen corneal treatment zone is substantially equivalent to a pupil aperture.

16. Apparatus for identifying corneal tissue to be ablated, comprising:
means for determining an initial surface around a visual axis of an eye;
means for determining a visual system error of said eye;
means for applying said visual system error to said initial surface to determine a target surface;
means for offsetting said initial surface and said target surface each by an amount substantially equal to an offset of said visual axis from said pupil center; and
means for subtracting said target surface from said initial surface to establish ablation values.

17. The apparatus for identifying corneal tissue to be ablated according to claim 16, further comprising:
means for locating said visual axis of said eye.

18. The apparatus for identifying corneal tissue to be ablated according to claim 17, further comprising:
means for locating said pupil center of said eye.

19. The apparatus for identifying corneal tissue to be ablated according to claim 16, wherein said initial surface and said target surface each comprise:
a keratometric surface.

20. The apparatus for identifying corneal tissue to be ablated according to claim 16, wherein said initial surface comprises:
a wavefront surface.

21. The apparatus for identifying corneal tissue to be ablated according to claim 16, wherein:
said wavefront surface is a piecewise refractive surface.

22. The apparatus for identifying corneal tissue to be ablated according to claim 16, wherein:
said means for subtracting determines said ablation values along a diameter of a chosen corneal treatment zone.

23. The apparatus for identifying corneal tissue to be ablated according to claim 22, further comprising:
means for ensuring that said ablation values determined along said diameter of said chosen corneal treatment zone are positive thereby defining a resulting ablation profile.

24. The apparatus for identifying corneal tissue to be ablated according to claim 23, further comprising:
means for using said resulting ablation profile as a guide for corneal tissue to be ablated, with greater positive values defining deeper ablation depths.

25. The apparatus for identifying corneal tissue to be ablated according to claim 23, wherein said means for ensuring comprises:
means for setting to zero any negative ones of said established ablation values.

26. The apparatus for identifying corneal tissue to be ablated according to claim 16, wherein:
said means for determining said initial surface utilizes a topographic keratometer.

27. The apparatus for identifying corneal tissue to be ablated according to claim 16, wherein:
said means for determining said visual system error utilizes a phoropter.

28. The apparatus for identifying corneal tissue to be ablated according to claim 16, wherein:
said means for determining said visual system error utilizes an auto-refractor device.

29. The apparatus for identifying corneal tissue to be ablated according to claim 16, wherein said means for applying said visual system error to said initial surface comprises:
means for adding said visual system error to said initial surface.

30. The apparatus for identifying corneal tissue to be ablated according to claim 22, wherein:
said diameter of said chosen corneal treatment zone is substantially equivalent to a pupil aperture.
